(19) United States
(12) Patent Application Publication
(10) Pub. No.: US 2006/0241037 A1
(43) Pub. Date: Oct. 26, 2006

(54) Compositions comprising trefoil factor family peptides and/or mucoadhesives and proton pump ihhibitor prodrugs

(75) Inventors: Orest Olejnik, Coto de Caza, CA (US); Peter M. Bakhit, Huntington Beach, CA (US); Richard Graham, Irvine, CA (US); Jie Shen, Irvine, CA (US); Devin F. Welty, Foothill Ranch, CA (US); Diane D. Tang-Liu, Newport Beach, CA (US)

Correspondence Address: ALLERGAN, INC., LEGAL DEPARTMENT, 2525 DUPONT DRIVE, T2-7H, IRVINE, CA, 92612-1599, US

(73) Assignee: ALLERGAN INC., Irvine, CA (US)

(21) Appl. No.: 10/568,330

(22) PCT Filed: Aug. 25, 2004
(86) PCT No.: PCT/US04/27776

§ 371 Date: Feb. 15, 2006

Related U.S. Application Data
(60) Provisional application No. 60/508,416, filed on Oct. 3, 2003.

(51) Int. Cl. A61K 38/17; A61K 31/4439

(52) U.S. Cl. 514/12; 514/338

(57) ABSTRACT

Disclosed are methods of preventing or treating a disease or adverse condition affecting the gastrointestinal tract of a mammal which comprise orally administering to a mammal a therapeutically effective amount of a prodrug of a proton pump inhibitor and an effective amount of a trefoil family factor peptide, mucoadhesive agent, or a combination thereof. Also disclosed are compositions which are suitable for use in a pharmaceutical dosage form. These compositions comprise a prodrug of a proton pump inhibitor, and also comprise a trefoil family factor peptide, a mucoadhesive component, or a combination thereof.

### FIELD OF THE INVENTION

The present invention relates to pharmaceutical compositions and methods. In particular, the present invention relates to pharmaceutical compositions and methods related to treating gastric disorders.

### BACKGROUND OF THE INVENTION

### Description of Related Art

Benzimidazole derivatives intended for inhibiting gastric acid secretion are disclosed in U.S. Pat. Nos. 4,045,563; 4,255,431; 4,628,098; 4,686,230; 4,758,579; 4,965,269; 5,021,433; 5,430,042 and 5,708,017. Generally speaking, the benzimidazole-type inhibitors of gastric acid secretion are believed to work by undergoing a rearrangement to form a thiophilic species which then covalently binds to gastric H,K-ATPase, the enzyme involved in the final step of proton production in the parietal cells, and thereby inhibits the enzyme. Compounds which inhibit the gastric H,K-ATPase enzyme are generally known in the field as “proton pump inhibitors” (PPI).

Some of the benzimidazole compounds capable of inhibiting the gastric H,K-ATPase enzyme have found substantial use as drugs in human medicine and are known under such names as LANSOPRAZOLE (U.S. Pat. No. 4,628,098), OMEPRAZOLE (U.S. Pat. Nos. 4,255,431 and 5,693,818), ESOMEPRAZOLE (U.S. Pat. No. 6,369,085) PANTOPRAZOLE (U.S. Pat. No. 4,758,579), and RABEPRAZOLE (U.S. Pat. No. 5,045,552). Some of the diseases treated by proton pump inhibitors and specifically by the five above-mentioned drugs include peptic ulcer, heartburn, reflux esophagitis, erosive esophagitis, non-ulcer dyspepsia, infection by Helicobacter pylori, alrynitis and asthma.

Whereas the proton pump inhibitor type drugs represent a substantial advance in the field of human and veterinary medicine, they are not totally without shortcomings or disadvantages. For example, it is believed that the short systemic half-life of the drug limits the degree of gastric acid suppression currently achieved. Furthermore, it appears that the short plasma half-life of the drug may contribute to significant gastric pH fluctuations that occur a several times a day in patients undergoing PPI therapy. Additionally, PPIs are acid-labile, and in most cases it is necessary to enterically coat the drug in order to prevent the acidic milieu of the stomach from destroying the drug before it can act. Thus, any contribution that might improve the plasma half-life of the presently used proton pump inhibitors will be a significant improvement in the art.

As further pertinent background to the present invention, applicants note the concept of prodrugs which is well known in the art. Generally speaking, prodrugs are derivatives of per se drugs, which after administration undergo conversion to the physiologically active species. The conversion may be spontaneous, such as hydrolysis in the physiological environment, or may be enzyme catalyzed. From among the voluminous scientific literature devoted to prodrugs in general, the foregoing examples are cited: Design of Prodrugs (Bundgaard H. ed.) 1985 Elsevier Science Publishers B. V. (Biomedical Division), Chapter 1; Design of Prodrugs: Bioreversible derivatives for various functional groups and chemical entities (Hans Bundgaard); Bundgaard et al. Int. J. of Pharmaceutics 22 (1984) 45-56 (Elsevier); Bundgaard et al. Int. J. of Pharmaceutics 29 (1986) 19-28 (Elsevier); Bundgaard et al. J. Med. Chem. 32 (1989) 2503-2507 Chem. Abstracts 93, 137935y (Bundgaard et al.); Chem. Abstracts 95, 138493f (Bundgaard et al.); Chem. Abstracts 95, 138592n (Bundgaard et al.); Chem. Abstracts 110, 57664p (Alminger et al.); Chem. Abstracts 115, 64029s (Buur et al.); Chem. Abstracts 115, 189582y (Hansen et al.); Chem. Abstracts 117, 14347q (Bundgaard et al.); Chem. Abstracts 117, 55790x (Jensen et al.); and Chem. Abstracts 123, 17593b (Thomsen et al.).

A publication by Sih., et al. Journal of Medicinal Chemistry, 1991, vol. 34, pp 1049-1062, describes N-acyloxyakyl, N-alkoxycarbonyl, N-(aminoethyl), and N-alkoxyalkyl derivatives of benzimidazole sulfoxide as prodrugs of proton-pump inhibitors. According to this article these prodrugs exhibited improved chemical stability in the solid state and in aqueous solutions, but had similar activity or less activity than the corresponding parent compounds having a free imidazole N-H group. This publication does not provide data regarding the duration of the inhibitory activity of these prodrugs.

U.S. Pat. No. 6,093,734 and PCT Publication WO 00109498 (published on Feb. 24, 2000) describe prodrugs of proton pump inhibitors which include a substituted arylsulfonyl moiety attached to one of the benzimidazole nitrogens of proton pump inhibitors having the structure identical with or related to proton pump inhibitor drugs known by the names LANSOPRAZOLE, OMEPRAZOLE, PANTOPRAZOLE and RABEPRAZOLE.

PCT Publication WO 02/30920 describes benzimidazole compounds which are said to have gastric acid secretion inhibitory and anti H. pylori effects. PCT Publication WO 02/00166 describes compounds that are said to be nitric oxide (NO) releasing derivatives of proton pump inhibitors of the benzimidazole structure.

U.S. Pat. App. having the title “PRODRUGS OF PROTON PUMP INHIBITORS”, filed Jul. 15, 2003 by applicants Michael E. Garst, George Sachs, and Jai M. Shin, which has not yet been assigned a serial number, discloses prodrugs of the proton pump inhibitor type drugs having an arylsulfonyl group with an acidic functional group attached, which provided improved solubility in physiological fluids and improved cell penetration. These references, however do not mention that the properties of the gastrointestinal mucus layer would have an effect on the sustained release properties of PPIs and their prodrugs.

Trefoil peptides, or trefoil factor family (TFF) peptides are a class of peptides which comprise a common structural motif, known as the trefoil domain, as part of their structure. The trefoil motif comprises about 20 to about 60 amino acid residues (usually about 40) containing six cysteine residues. The six cysteine residues form three disulfide bridges that complete three loops in the peptide chain so that the roughly 40 residues have a clover-like shape, known as the trefoil domain. TFF-peptides can have one or two trefoil domains per molecule, and may comprise additional amino acid residues which are not part of the trefoil domain. To date, three types of TFF-peptides have been isolated from humans-TFF1 (also known as pS2), TFF2 (also known as SP), and TFF3 (also known as rIF). TFF1 and TFF3 peptides each contain one trefoil domain, while TFF2 peptides contain two trefoil domains. TFF1 and TFF2 peptides are both produced by mucus-producing cells of stomach, while TFF3 peptides are produced by goblet cells of small and large intestine.

All three forms of TFF-peptides are known to be produced in epithelial cells around areas of damage to mucus membrane, suggesting that trefoils have a role in healing injury, particularly to epithelial cells. It is believed that TFF-peptides assist healing by both stabilizing mucus membrane at the injury site and by stimulating repair. It has been shown that TFF-peptides noncovalently link mucin, thus influencing the rheology (e.g. increases viscosity) of mucus gels. [Hauser F, Poulsom R, Chinery R, et al, Proc Natl Acad Sci USA, 1993, vol. 90, pp. 6961-6965; and Babyatsky M W, deBeaumont M, Thim L, Podolky D K, Gastroenterology, 1996, vol. 110, pp. 489-497]. TFF-peptides also appear to be responsible for promoting the migration of epithelial cells to the site of injury, thus stimulating repair. [Goke M, et al, Experimental Cell Research, 2001, vol 264, pp. 337-344; and Playford R J, Journal of the Royal College of Physicians of London, vol 31, pp. 37-40]

Mucoadhesives are well known in the art as compounds or compositions of matter that are capable of adhering to mucus membranes. A number of mucoadhesive compositions are known to help to stabilize and increase the viscosity of mucus (Madsen, Flemming; Eberth, Kirsten; and Smart, John D.; Journal of Controlled Release (1998), 50(1-3), 167-178; and Foster, S. N. E.; Pearson, J. P.; Hutton, D. A.; Allen, A.; Dettmar, P. W.; Clinical Science (1994), 87(6), 719-26), and have been suggested to be useful in treating gastric disorders associated with compromised mucus membrane. The stabilization of mucus membrane by mucoadhesives is believed to be at least partially due to noncovalent interactions between the mucoadhesive and mucin which serve to link the molecules together, thus reinforcing mucus membrane structure and enhancing viscosity. However, to the best of our knowledge, no reference has suggested that modification of the mucus properties of the stomach using mucoadhesives would be related to sustained delivery of a PPI or related compounds.

The description of the related art provided herein is given merely to point out how the present invention is related to the current art and to provide guidance in practicing the invention. However, one should not construe the statements made above as making any determination or conclusion whatever concerning whether any of the references cited herein is prior art.

### SUMMARY OF THE INVENTION

Disclosed herein are methods of preventing or treating a disease or adverse condition affecting the gastrointestinal tract of a mammal. These methods comprise orally administering to a mammal a therapeutically effective amount of a prodrug of a proton pump inhibitor. An effective amount of a trefoil family factor peptide, mucoadhesive agent, or a combination thereof is also administered to the mammal.

Other methods of preventing or treating a disease or adverse condition are also disclosed herein. These methods comprise administering directly into the gastrointestinal tract of a mammal an effective amount of a therapeutically active agent and a therapeutically effective amount of a trefoil factor family peptide. The therapeutically active agent administered in these methods comprises a compound which, when administered orally, results in inhibition of the gastric H,K-ATPase enzyme. Additionally, the disease or adverse condition being prevented or treated by this method affects the gastrointestinal tract.

Also disclosed are compositions which are suitable for use in a pharmaceutical dosage form. These compositions comprise a prodrug of a proton pump inhibitor, and also comprise a trefoil family factor peptide, a mucoadhesive component, or a combination thereof.

Oral dosage forms comprising a therapeutically active component and a trefoil factor family peptide are also disclosed herein. In these dosage forms said therapeutically active component is selected from the group consisting of proton pump inhibitors, prodrugs of proton pump inhibitors, and combinations thereof.

### BRIEF DESCRIPTION OF THE FIGURES

FIG. 1 is a plot of the systemic T_{1/2} of proton pump inhibitors omeprazole and lansoprazole, following oral administration of their corresponding prodrugs in dog, as a function of membrane permeability of the prodrugs, measured as the permeability coefficient (Papp) across Caco-2 cells in the apical to basolateral direction.

### DETAILED DESCRIPTION OF THE INVENTION

While not intending to be bound in any way by theory, we have surprisingly discovered that thickening the mucus layer of the gastrointestinal tract in conjunction with administration of a proton pump inhibitor (PPI) or a prodrug of a proton pump inhibitor will improve the sustained systemic delivery of PPIS. We have surprisingly found that the oral administration of PPIs or their prodrugs in compositions or by methods disclosed herein will increase the systemic half-life of the proton pump inhibitors relative to oral administration of the proton pump inhibitor. While not intending to be bound in any way by theory, it is believed that oral administration of a PPI as disclosed herein will increase the systemic half-life of the proton pump inhibitor by reducing the rate of absorption of the PPI or its related prodrug from the gastrointestinal tract into the bloodstream.

Certain embodiments relate to methods of preventing or treating a disease or adverse condition. In one embodiment, the disease or adverse condition affects the gastrointestinal tract of a mammal. These methods comprise orally administering to a mammal a therapeutically effective amount of a prodrug of a proton pump inhibitor. An effective amount of a trefoil family factor peptide, a mucoadhesive, agent, or a combination thereof is also administered to the mammal. One embodiment comprises orally administering a therapeutically effective amount of a prodrug of a proton pump inhibitor and an effective amount of a trefoil factor family peptide. Another embodiment relates to orally administering a therapeutically effective amount of a prodrug of a proton pump inhibitor and an effective amount of a mucoadhesive agent. Another embodiment comprises orally administering a therapeutically effective amount of a prodrug of a proton pump inhibitor and an effective amount of a trefoil factor family peptide and an effective amount of a mucoadhesive agent. The proton pump inhibitor and the prodrug of the proton pump inhibitor may also be combined in conjunction with any of the above described embodiments. Additionally, a combination of two or more proton pump inhibitors may also be used in conjunction with any of the above described embodiments.

In another embodiment, these methods comprise administering directly into the gastrointestinal tract of a mammal an effective amount of a therapeutically active agent and a therapeutically effective amount of a trefoil factor family peptide. The therapeutically active agent administered in these methods comprises a compound which, when administered orally, results in inhibition of the gastric H,K-ATPase enzyme. In this embodiment the therapeutically active agent is any compound which directly inhibits the gastric H,K-ATPase enzyme, or any compound which decomposes in any part of the body after administration into any chemical species which directly inhibits the gastric H,K-ATPase enzyme. Additionally, the disease or adverse condition being prevented or treated by this method affects the gastrointestinal tract.

Other embodiments relate to compositions which are suitable for use in a pharmaceutical dosage form. These compositions comprise a prodrug of a proton pump inhibitor, and also comprise a trefoil family factor peptide, a mucoadhesive component, or a combination thereof. One composition comprises a therapeutically effective amount of a prodrug of a proton pump inhibitor and an effective amount of a trefoil factor family peptide. Another composition comprises a therapeutically effective amount of a prodrug of a proton pump inhibitor and an effective amount of a mucoadhesive agent. Another composition comprises a therapeutically effective amount of a prodrug of a proton pump inhibitor and an effective amount of a trefoil factor family peptide and an effective amount of a mucoadhesive agent. The proton pump inhibitor and the prodrug of the proton pump inhibitor may also be used together in conjunction with any of the above described compositions. Additionally, a combination of two or more proton pump inhibitors may also be used in conjunction with any of the above described compositions.

Oral dosage forms comprising a therapeutically active component and a trefoil factor family peptide are also disclosed herein. In these dosage forms said therapeutically active component is selected from the group consisting of proton pump inhibitors, prodrugs of proton pump inhibitors, and combinations thereof. One type of dosage form comprises a proton pump inhibitor and a trefoil factor family peptide. Another type of dosage form comprises a prodrug of a proton pump inhibitor and a trefoil factor family peptide. Another type of dosage form comprises both a proton pump inhibitor and a prodrug of a proton pump inhibitor and a trefoil factor family peptide. Another type of dosage form comprises two or more prodrugs of a proton pump inhibitor and a trefoil factor family peptide. In other dosage forms, a mucoadhesive is used in conjunction with the therapeutically active agent and the trefoil factor family peptide.

The term “proton pump inhibitor” as used herein has the meaning previously described. Methods of preparing proton pump inhibitors are well known in the art. Although the term proton pump inhibitor is to be interpreted broadly in accordance with the definition provided herein. Certain embodiments relate to particular proton pump inhibitors. In one embodiment, the proton pump inhibitor is selected from the group consisting of omeprazole, esomeprazole, lansoprazole, pantoprazole, and rabeprazole. In another embodiment, the proton pump inhibitor is omeprazole. In another embodiment, the proton pump inhibitor is esomeprazole. In another embodiment, the proton pump inhibitor is pantoprazole. In another embodiment, the proton pump inhibitor is rabeprazole. In another embodiment, the proton pump inhibitor is lansoprazole.

The term “prodrug” as used herein has the meaning previously described and refers to a prodrug of a proton pump inhibitor. Methods of preparing these prodrugs are described in U.S. Pat. No. 6,093,734; and U.S. patent application Ser. No. 09/783,807, filed Feb. 14, 2001, incorporated herein by reference. However, these methods are only given to provide guidance, and are not meant to limit the scope of the invention in any way. One of ordinary skill in the art will recognize that there are many ways in which the prodrugs of the present invention can be prepared without departing from the spirit and scope of the present invention. Thus, the prodrugs disclosed herein are compounds which will be converted a proton pump inhibitor after being administered to an individual. Thus, for certain embodiments, the prodrug is converted to omeprazole, esomeprazole, lansoprazole, pantoprazole, oral rabeprazole after oral administration. In other embodiments, the prodrug is converted to omeprazole after oral administration. In other embodiments, the prodrug is converted to lansoprazole after oral administration.

Prodrugs may comprise any pharmaceutically acceptable salts and still retain their identity as prodrugs, since under biological conditions acidic or basic groups will be found in the form required by the pH of the particular environment the molecule is in and by the acidity or basicity of the functional group in question. Thus for example, a both prodrug comprising a carboxylic acid, and its pharmaceutically acceptable salts are properly understood to be prodrugs.

A “pharmaceutically acceptable salt” is any salt that retains the activity of the parent compound and does not impart any deleterious or untoward effect on the subject to which it is administered and in the context in which it is administered.

Pharmaceutically acceptable salts of acidic functional groups may be derived from organic or inorganic bases. The salt may be a mono or polyvalent ion. Of particular interest are the inorganic ions, lithium, sodium, potassium, calcium, and magnesium. Organic salts may be made with amines, particularly ammonium salts such as mono-, di- and trialkyl amines or ethanol amines. Salts may also be formed with caffeine, tromethamine and similar molecules. Hydrochloric acid or some other pharmaceutically acceptable acid may form a salt with a compound that includes a basic group, such as an amine or a pyridine ring.

In some circumstances the prodrugs have specific structural features. In one embodiment, the prodrug comprises a sulfonyl moiety, and said prodrug is converted to omeprazole after oral administration. The term “sulfonyl moiety” refers to an SO₂ moiety, where the sulfur is bonded to two additional atoms beside the two oxygen atoms. In another embodiment, the prodrug comprises a sulfonyl moiety and said prodrug is converted to lansoprazole after oral administration.

In relation to a prodrug, the term “sulfonyl leaving group” used herein refers to a biologically labile moiety which has an SO₂ functional group. The sulfonyl leaving group is cleaved from the remainder of the molecule under biological conditions at the bond between the sulfur atom of the SO₂ functional group and the active part of the molecule. The active part of the molecule is the proton pump inhibitor; a species that is converted to the proton pump inhibitor by protonation, deprotonation, tautomerization, or a similar process; or is a reactive intermediate that is readily converted to the proton pump inhibitor. In another embodiment the therapeutically active component comprises a prodrug having a sulfonyl leaving group, wherein said sulfonyl leaving group also comprises a carboxylic acid moiety or a pharmaceutically acceptable salt thereof.

In certain embodiments the therapeutically active agent comprises a benzimidazole derivative. A benzimidazole derivative is defined as a compound having a core benzimidazole structure with one or more attached substituent groups. While not intending to limit the scope of claims in any way, particularly useful substituents comprise moieties such as sulfoxy, alkyl, alkoxy, fluoroalkyl, fluoroalkoxy, and sulfonyl.

Other embodiments comprise a benzimidazole derivative and a biological leaving group. The term “biological leaving group” as used herein refers to a moiety which is cleaved from the remainder of the molecule in the body of a mammal such that the remainder of the molecule is a proton pump inhibitor, or is readily converted to a proton pump inhibitor by a process such a protonation; deprotonation; quenching of an unstable intermediate such as a radical, radical ion, carbocation, carbene, or nitrene; tautomerization; or a similar process. In certain embodiments, the biological leaving group comprises a sulfonyl moiety. In other embodiments, the biological leaving group further comprises a carboxylic acid or a pharmaceutically acceptable salt thereof.

In other embodiments the prodrug comprises 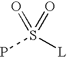
or a pharmaceutically acceptable salt thereof;
wherein
the dashed line indicates a bond that is broken systemically in said mammal;
P is a moiety that is converted systemically to a proton pump inhibitor as a result of cleavage of the bond indicated by the dashed line; and
L is a moiety which comprises a carboxylic acid.

In relation to this embodiment, the cleavage of the bond indicated by the dashed line occurs during or after absorption of the prodrug from the gastrointestinal tract into the blood. P is not necessarily a proton pump inhibitor per se, but is a species which can be converted to a proton pump inhibitor by such elementary reactions as acid-base type reactions, tautomerization, or similar processes. P may also be a reactive intermediate such as a cation, anion, radical ion, carbene, nitrene, or similar species, which is rapidly converted into the proton pump inhibitor in the body of the individual receiving the prodrug.

In certain embodiments related to the above disclosed structure, L comprises a phenyl moiety. In other embodiments, P is converted systemically to a proton pump inhibitor selected from the group consisting of omeprazole, esomeprazole, lansoprazole, pantoprazole, and rabeprazole. In other embodiments, P is converted systemically to omeprazole. In other embodiments, P is converted systemically to lansoprazole.

In other embodiments, the prodrug has a structure comprising 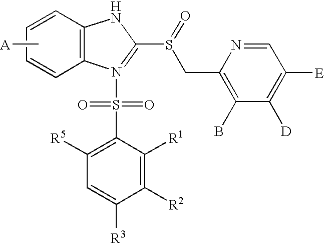
or a pharmaceutically acceptable salt thereof
wherein
A is H, OCH₃, or OCHF₂;
B is CH₃ or OCH₃;
D is OCH₃, OCH₂CF₃, or O(CH₂)₃OCH₃;
E is H or CH₃;
R¹, R², R³, and R⁵ are independently H, CH₃, CO₂H, CH₂CO₂H, (CH₂)₂CO₂H, CH(CH₃)₂, OCH₂C(CH₃)₂CO₂H, OCH₂CO₂CH₃, OCH₂CO₂H, OCH₂CO₂NH₂, OCH₂CONH₂(CH₂)₅CO₂CH₃, or OCH₃.

In other embodiments related to that described above, R¹, R², R³, and R⁵ are independently H, CH₃, CO₂H, CH₂CO₂H, (CH₂)₂CO₂H, OCH₂CO₂CH₃, OCH₂CO₂H, OCH₂CONH₂(CH₂)₅CO₂CH₃, or OCH₃.

The term “membrane permeability” used in relation to this invention refers to the value obtained by carrying out the procedure described in Example 1 herein. While not intending to limit the scope of the invention in any way, it is believed that the membrane permeability obtained by the procedure of Example 1 is a good relative quantitative measurement of the ability of a given compound to diffuse through a membrane in a living system such as the gastrointestinal lining of a human or another mammal. While not intending to be bound in any way by theory, although a direct correlation between the two properties may not necessarily be made, the relative trend in membrane permeability between compounds in a series appears to be consistent with the relative trend in the ability of the compounds in a series to pass through the gastrointestinal lining.

In some embodiments, the membrane permeability of the proton pump inhibitor is more than twice the membrane permeability of the prodrug. In other embodiments, the membrane permeability of the proton pump inhibitor is more than 10 times the membrane permeability of the prodrug. In other embodiments, the membrane permeability of the proton pump inhibitor is more than 100 times the membrane permeability of the prodrug. In other embodiments, the membrane permeability of the proton pump inhibitor is more than 150 times the membrane permeability of the prodrug.

In the case that the therapeutically active component is a single compound, certain embodiments relate to the value of the membrane permeability for the therapeutically active component. In one embodiment the therapeutically active component has a membrane permeability which is less than 1.4×10⁻⁵ cm/sec. In another embodiment the therapeutically active component has a membrane permeability which is less than 1×10⁻⁶ cm/sec. In another embodiment the therapeutically active component has a membrane permeability which is less than 5×10⁻⁷ cm/sec. In another embodiment the therapeutically active component has a membrane permeability which is less than 1×10⁻⁷ cm/sec. In another embodiment the therapeutically active component has a membrane permeability which is less than 5×10⁻⁸ cm/sec.

Other embodiments comprise both a proton pump inhibitor and the prodrug of the proton pump inhibitor. In certain of these embodiments, the membrane permeability of the proton pump inhibitor is more than 10 times the membrane permeability of the prodrug. In other embodiments, the membrane permeability of the proton pump inhibitor is more than 100 times the membrane permeability of the prodrug. In other embodiments, the membrane permeability of the proton pump inhibitor is more than 150 times the membrane permeability of the prodrug.

Other embodiments relate to mixtures of prodrugs. In circumstances that two prodrugs of a proton pump inhibitor are used in the methods or compositions described herein, the prodrugs, certain embodiments relate to the membrane permeability ratio for the two prodrugs. The membrane permeability ratio in relation to these embodiments is defined as the value of the membrane permeability of the prodrug having the higher membrane permeability, divided by the membrane permeability of the prodrug having the lower membrane permeability. In one embodiment, the prodrugs have a membrane permeability ratio of from 2 to 1000. In another embodiment, the prodrugs have a membrane permeability ratio of from 10 to 500. In another embodiment, the prodrugs have a membrane permeability ratio of from 100 to 500.

The term trefoil factor family (TFF) peptide as used herein refers to any peptide, whether natural or synthetic, which comprises the trefoil motif described previously herein. That is, the TF-peptide comprises a residue comprising from 20 to about 60 amino acids, including six cysteine residues. The cysteine residues form disulfide bonds which cause the peptide residue to have a clover-like shape comprising three loops. The methods of preparing of TFF-peptides, such as recombinant expression of peptides and synthetic peptide synthesis, are well known in the art. For example, methods of preparing TFF-peptides are included in the following references: U.S. Pat. No. 6,525,018; Allen, et. al., J Clin Gastroenterol 1998; 10 (Suppl 1): S93-S98; Ligumsky, et. al., Isr J Med Sci 1986; 22:801-806; Dignass, et. al., J. Clin. Invest., 94, 376-383; Babyatsky, et. al., Gastroenterology, 110,489-497; Hauser, et. al., Proc. Natl. Acad. Sci. USA, vol. 90, pp. 6961-6965, August 1993; WO 02102403; and WO 02085402, incorporated herein by reference. In one embodiment the trefoil factor family peptide is TFF1, TFF2, or TFF3. In another embodiment the trefoil factor family peptide is TFF1 or TFF2. In another embodiment the trefoil factor family peptide is TFF1. In another embodiment the trefoil factor family peptide is TFF2. In another embodiment the trefoil factor family peptide is TFF3.

With respect to this invention, the term “mucoadhesive” means a natural or synthetic component, including macromolecules, polymers, and oligomers, or mixtures thereof, that can adhere to a subject's mucous membrane. Adhesion of mucoadhesives to the mucous membrane occurs primarily through noncovalent interactions, such as hydrogen bonding and Van der Waal forces (Tabor et al., 1977 J. Colloid Interface Sci. 58:2 and Good 1977 J. Colloid Interface Sci. 59:398). Examples of mucoadhesives for use in the embodiments disclosed herein include, but are not limited to, Carbopol®, pectin, alginic acid, alginate, chitosan, hyaluronic acid, polysorbates, such as polysorbate-20, -21, -40, -60, -61, -65, -80, -81, -85; poly(ethyleneglycol), such as PEG-7, -14, -16, -18, -55, -90, -100, -135, -180, -4, -240, -6, -8, -9, -10, -12, -20, or -32; oligosaccharides and polysaccharides, such as Tamarind seed polysaccharide, gellan, carrageenan, xanthan gum, gum Arabic, and dextran; cellulose esters and cellulose ethers; modified cellulose polymers, such as carboxymethylcellulose, hydroxyethylcellulose, hydroxypropyl methylcellulose, hydroxyethyl ethylcellulose; polyether polymers and oligomers, such as polyoxyethylene; condensation products of poly(ethyleneoxide) with various reactive hydrogen containing compounds having long hydrophobic chains (e.g. aliphatic chains of about 12 to 20 carbon atoms), for example, condensation products of poly(ethylene oxide) with fatty acids, fatty alcohols, fatty amides, polyhydric alcohols; polyether compounds, such as poly(methyl vinyl ether), polyoxypropylene of less than 10 repeating units; polyether compounds, such as block copolymers of ethylene oxide and propylene oxide; mixtures of block copolymers of ethylene oxide and propylene oxide with other excipients, for example poly(vinyl alcohol); polyacrylamide; hydrolyzed polyacrylamide; poly(vinyl pyrrolidone); poly(methacrylic acid); poly(acrylic acid) or crosslinked polyacrylic acid, such as Carbomer®, i.e., a homopolymer of acrylic acid crosslinked with either an allyl ether of pentaerythritol, an allyl ether of sucrose, or an allyl ether of propylene. In certain embodiments the mucoadhesive is a polysaccharide. One polysaccharide which is particularly useful as a mucoadhesive in the embodiments disclosed herein is Tamarind seed polysaccharide, which is a galactoxyloglucan that is extracted from the seed kernel of Tamarindus Indica, and can be purchased from TCI America of Portland, Oreg.

While not intending to limit the scope of the invention in any way, or to be bound in any way by theory, it is believed that there will be a strong synergy present between the PPI or related prodrug and the thickened or enhanced mucus membrane related to the use of the mucoadhesive and/or TFF-peptide. We have found that the rate of passage of a PPI through the gastrointestinal tract to the bloodstream is highly sensitive to structural modifications to the PPI, which has a significant impact upon the systemic half-life of the proton pump inhibitor. While not intending to be bound in any way by theory, this suggests that the absorption rate of PPIs and related compounds through the lining of the gastrointestinal tract is an important factor in determining the overall pharmacokinetics of these compounds. It follows that the systemic half-life will be sensitive to changes which affect the rate of passage of the molecule through the lining of the gastrointestinal tract. Thus, while not intending to be bound in any way by theory, the systemic half-life of the PPI should be enhanced by administration of a compound or composition that retards the passage of the molecule through the gastrointestinal lining. It is believed that the mucus membrane is a protective barrier for the gastrointestinal lining, it follows that any reinforcement of the structure of the mucus membrane or any increase in the amount or viscosity of the mucus will retard passage to the gastrointestinal lining, and thus through the gastrointestinal lining. Additionally, since mucin is a glycoprotein, it has both hydrophobic and hydrophilic domains. Thus, while not intending to be bound in any way by theory, this suggests that mucin can bind to both the hydrophobic and hydrophilic parts of the drug and act as a depot for sustained release of the drug, increasing the bioavailability of the drug while slowing its absorption. Additionally, any repair or reinforcement of mucin will help prevent or heal damage that may have occurred as a result of the gastric condition.

The best mode of making and using the present invention are described in the following examples. These examples are given only to provide direction and guidance in how to make and use the invention, and are not intended to limit the scope of the invention in any way.

### Test Compounds

Membrane permeability and oral bioavailability tests were carried out for the compounds shown in Table 1 below. The generic structure, I, is shown as a combination of a proton pump inhibitor (X) and a sulfonyl-bearing moiety which is attached to the proton pump inhibitor to form the prodrug according to the formula below. The identity of each group represented by R¹-R⁵ is shown in the table.

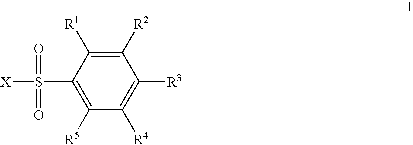

The different possibilities for X are shown below.

TABLE 1

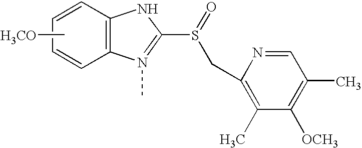

OME

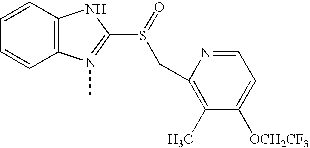

LNZ

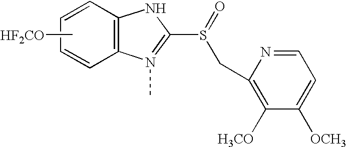

PNT

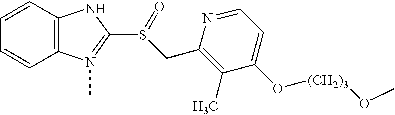

RAB

| Compound | X | R¹ | R² | R³ | R⁴ | R⁵ |
|---|---|---|---|---|---|---|
| 1 | OME | H | H | OCH₂CO₂H | H | H |
| 2 | OME | CH₃ | H | OCH₂CO₂H | H | CH₃ |
| 3 | OME | H | H | OCH₂C(CH₃)₂CO₂H | H | H |
| 4 | OME | CH₃ | H | OCH₂C(CH₃)₂CO₂H | H | CH₃ |
| 5 | OME | H | H | CH₂CO₂H | H | H |
| 6 | OME | H | CO₂H | H | H | H |
| 7 | LNZ | H | CO₂H | H | H | H |
| 8 | LNZ | H | CO₂H | OCH₃ | H | H |
| 9 | LNZ | H | H | CH₂CO₂H | H | H |
| 10 | LNZ | H | H | OCH₂CO₂H | H | H |
| 11 | LNZ | H | H | OCH₂C(CH₃)₂CO₂H | H | H |
| 12 | LNZ | H | CH₂CO₂H | CH₂CO₂H | H | H |
| 13 | LNZ | H | CO₂H | H | H | CH₃ |
| 14 | LNZ | H | CO₂H | H | H | OCH₃ |
| 15 | LNZ | CH(CH₃)₂ | H | CH₂CO₂H | H | H |
| 16 | LNZ | H | OCH₂CO₂H | CO₂H | H | H |
| 17 | LNZ | CH(CH₃)₂ | H | OCH₂CO₂H | H | CH₃ |
| 18 | LNZ | H | H | CO₂H | H | H |
| 19 | LNZ | H | (CH₂)₂CO₂H | CH₃ | H | H |
| 20 | OME | H | H | OCH₂CO₂CH₃ | H | H |
| 21 | OME | H | H | OCH₂CO₂NH₂ | H | H |
| 22 | OME | H | CO₂H | CO₂H | H | H |
| 23 | OME | H | CO₂H | OCH₂CO₂H | H | H |
| 24 | OME | H | OCH₂CO₂H | OCH₂CO₂H | H | H |
| 25 | OME | OCH₃ | H | CO₂H | H | H |
| 26 | OME | H | | CO₂H | H | H |
| 27 | OME | H | CO₂H | H | H | CH₃ |
| 28 | PNT | H | H | OCH₂CO₂H | H | H |
| 29 | PNT | H | CO₂H | H | H | CH₃ |
| 30 | RAB | H | CO₂H | H | H | H |
| 31 | RAB | H | CO₂H | H | H | CH₃ |
| 32 | RAB | CH₃ | H | OCH₂CO₂H | H | CH₃ |
| 33 | RAB | H | H | CO₂H | H | H |
| 34 | LNZ | CH₃ | H | OCH₂CO₂H | H | CH₃ |
| 35 | LNZ | H | OCH₂CO₂H | OCH₂CO₂H | H | H |
| 36 | LNZ | H | H | CO₂H | H | H |
| 37 | LNZ | CH₃ | H | CO₂H | H | H |
| 38 | LNZ | H | (CH₂)₂CO₂H | OCH₃ | H | H |
| 39 | OME | CH₃ | H | OCH₂CONH₂(CH₂)₅CO₂CH₃ | H | CH₃ |
| 40 | OME | H | H | OCH₂CONH₂(CH₂)₅CO₂CH₃ | H | H |
| 41 | OME | H | H | (CH₂)₂CO₂H | H | H |
| 42 | OME | H | (CH₂)₂CO₂H | OCH₃ | H | H |

Compounds were prepared according to procedures described the U.S. Pat. App. having the title “PRODRUGS OF PROTON PUMP INHIBITORS”, filed Jul. 15, 2003 by applicants Michael E. Garst, George Sachs, and Jai M. Shin, which has not yet been assigned a serial number; and the U.S. Pat. App. having the title “PROCESS FOR PREPARING ISOMERICALLY PURE PRODRUGS OF PROTON PUMP INHIBITORS”, filed Jul. 15, 2003 by applicants Michael E. Garst, Lloyd J. Dolby, Shervin Esfandiari, Vivian R. Mackenzie, Alfred A. Avey, Jr., David C. Muchmore, Geoffrey K. Cooper, and Thomas C. Malone, which has not yet been assigned a serial number, incorporated by reference previously herein.

Omeprazole and lansoprazole were purchased from Sigma (St. Louis, Mo.).

### EXAMPLE 1

Determination of membrane permeability in all examples described herein was accomplished by the following procedure. This procedure is also used to determine whether a given prodrug falls within the scope of those claims given herein which relate to membrane permeability.

Materials/Methods

Test System: Cultured Caco-2 cells
Seeding Density: 2×10⁵ cells/cm² in Costar 12 well Transwell™ plates
Culture Age: 17-21 days post seeding
Source: American Type Culture Collection, Manassas, Va.
Growth Media: Dulbecco's Modified Eagle Media (DMEM) (Gibco BRL) supplemented with 10% fetal bovine serum and 0.1% nonessential amino acids
Dosing Formulation: 10 μM proton pump inhibitor or prodrug in DMEM. Make on the day of dosing.
Assay: LC-MS/MS
   Bi-Directional Transport Experiment:

Caco-2 cells were seeded on Costar™ 12 mm diameter, 0.4 μm pore size transwell filters, and were cultured at 37° C., 5% CO₂ in a humidified tissue culture chamber.

DMEM was equilibrated as a transport buffer in 37° C. water bath an hour before experiment. The cells were then equilibrated in transport buffer for 1 hr at 37° C.

Dosing solution (10 μM) was prepared by adding a 20 μL aliquot of a 10 mM stock solution of the prodrug to 20 mL of transport buffer.

Test Conditions:

Transport across Caco-2 cell monolayer was measured at 37° C., in the apical to basolateral direction (n=3).

Transport buffer was removed from both apical and basolateral compartment of filters. Dosing solution (0.2 mL) was added to the apical compartment of the cell layers on transwell filters, and 0.8 ml fresh pre-warmed transport buffer was added to basolateral compartment. Timing was started for transport, and at 5, 20, and 60 min after transport started, sample fluid (400 μL) was collected from the basolateral compartment. Fresh transport buffer (400 μL) was added back to the basolateral compartment, and the fluid was thoroughly mixed.

Transport samples, dosing solution, and standards (100 μL) each were mixed with 100 μl of a 500 ng/ml internal standard (Lansoprazole-D) for LC-MS/MS analysis, and part of each sample (100 μL) was vortexed and transferred into glass LC-MS/MS vials for analysis.

Data Analysis

The apparent permeability coefficient (Papp, cm/sec), otherwise known herein as the membrane permeability, is determined from the following relationship:
Papp=J/(ACₒ)
where J (pmol/min) is the transport rate, meaning the rate of prodrug movement through the cell layer, A (cm²) is the filter surface area, and Cₒ (μM) is the initial dosing concentration.
The transport rate J, is calculated as the slope of the linear regression fit for the transport amount over time data using Microsoft Excel® 97 SR-2 (Microsoft Corp. Redmond, Wash.),
Reference Standard:

Lucifer yellow (LY) was used as a paracellular permeability reference standard to determine integrity of cell layers used in the experiments. LY transport in the apical to basolateral direction was carried out in the same manner as described above. Fluorescence level in basolateral fluid sampled at 5, 20, and 60 min post dose was determined using Fluostar Galaxy (BMG Labtechnologies, Durham, N.C.) at excitation/emission wavelengths of 485/520 nm. A standard curve covering the range from 0.002 to 0.5 mg/mL is constructed to quantify the amount of LY in the transport sample to calculate permeability coefficient (Papp). Papp values below 1×10⁻⁶ cm/sec were considered acceptable and were used to normalize Papp values for test articles across experiments by multiplying the Papp values for the test articles by the factor x according to the following equation,
x=(1×10⁻⁶)/(S)
where S is the value of Papp obtained for LY.

### EXAMPLE 2

Oral bioavailability of omeprazole, lansoprazole, pantoprazole, rabeprazole, and test compounds was determined in rats (Sprague-Dawley) and dogs (beagle) by administering an oral solution to the animal and collecting serial blood samples through 24 hr post dose. Blood concentrations of the compounds omeprazole, lansoprazole, pantoprazole, rabeprazole, and test compounds were quantified using an achiral liquid chromatography tandem mass spectrometry method (LC-MS/MS). Systemic pharmacokinetic parameters were determined for omeprazole or lansoprazole using non-compartmental analysis in Watson® version 6.3, available from InnaPhase Corporation, Philadelphia, Pa. Results of the oral pharmacokinetic studies are presented in Tables 2A-2D below.

TABLE 2A
Systemic Omeprazole Half-life in Rats

| | | Equivalent | Systemic |
|---|---|---|---|
| Compound | Dosing | omeprazole | omeprazole |
| Administered | Route | dose (mg/kg) | half-life (hr) |
| Omeprazole | Oral | 10 | 0.31 |
| 1 | Oral | 10 | 1.7 |
| Omeprazole | Intravenous | 1 | 0.15 |
| 1 | Intravenous | 1 | 0.18 |

Table 2A shows the systemic half-life of omeprazole in rats after oral and intravenous administration of omeprazole and compound 1. Surprisingly, these results show that the systemic half-life of omeprazole after intravenous administration of omeprazole is nearly identical to that after intravenous administration of the prodrug (compound 1). The prodrug was not detected in the bloodstream 5 minutes after it was administered intravenously. These unexpected results demonstrate that in the case of compound 1, systemic conversion of the prodrug to omeprazole does not take an appreciable amount of time compared to the amount of time omeprazole is present systemically. By contrast, absorption of the prodrug from the gastrointestinal tract into the blood unexpectedly prolongs the systemic half-life of omeprazole to a significant extent relative to both the intravenous and oral administration of omeprazole. Table 2B shows a similar effect in dogs.

TABLE 2B
Systemic Omeprazole Half-life in Dogs

| | | Equivalent | Systemic |
|---|---|---|---|
| Compound | Dosing | omeprazole | omeprazole |
| Administered | Route | dose (mg/kg) | half-life (hr) |
| Omeprazole | Oral | 10 | 0.70 |
| 1 | Oral | 10 | 2.4 |
| Omeprazole | Intravenous | 1 | 0.60 |
| 1 | Intravenous | 1 | 1.0 |

Table 2C summarizes the systemic half-lives of the prodrugs and the PPIs for compounds 1-42 in dogs and rats. While not intending to be limited or bound in any way by theory, these results demonstrate that slow absorption of the prodrug from the gastrointestinal tract can contribute to an increase in the systemic half-life of the proton pump inhibitor. For many of the prodrugs in the table, the systemic half-life of the prodrug (i.e. the intact prodrug molecule) is either very short relative to the systemic half-life of the proton pump inhibitor, or is so short that the intact prodrug cannot be detected in the blood, and thus the half-life cannot be detected (NC). By contrast, however, for many of these same prodrugs, the measured systemic half-life of the proton pump inhibitor is significantly increased relative to the orally administered prodrug. Since the hydrolysis of the prodrugs in the blood does not contribute significantly to the increased systemic half-life of the proton pump inhibitors, it follows that the absorption of the prodrug from the gastrointestinal tract is slowed sufficiently to prolong the systemic half-life of the proton pump inhibitor. Thus, while not intending to be bound or limited in any way by theory, in the case of these particular prodrugs, it is the absorption step rather than the hydrolysis step that is the rate-limiting step of the pharmacokinetic process. In other words, the gastrointestinal tract, rather than the bloodstream, acts as the depot for the prodrug. Additionally, while not wishing to be limited or bound in any way by theory, since the absorption through the gastrointestinal tract is the rate-limiting pharmacokinetic step in the systemic consumption of the proton pump inhibitor for many of the compounds disclosed herein, the fact that the systemic half-life of these compounds varies widely supports the assertion that the rate of absorption of these compounds from the gastrointestinal tract is highly dependent upon the structure of the compound.

TABLE 2C
Systemic Half-Life of Prodrugs and PPIs in Dogs and Rats

| | Dog | | Rat | |
|---|---|---|---|---|
| | T_{1/2} | T_{1/2} | T_{1/2} | T_{1/2} |
| Compound | Prodrug | PPI | Prodrug | PPI |
| Omeprazole | | 0.696 (0.116) | | 0.308 |
| 1 | NC | 2.08 (1.19) | NC | 2.4 |
| 2 | 0.113 | 1.61 | | |
| | (n = 1) | | | |
| 3 | 0.311 | 0.813 | NC | 1.76 (0.93) |
| 4 | 1.26 | 0.837 | 0.342 | 0.708 (0.479) |
| 5 | 0.269 | 1.03 | NC | 1.7 |
| 6 | 0.303 | 1.91 | NC | 1.93 (0.39) |
| 20 | NC | 2.70 (0.62) | | |
| 21 | NC | 0.855 (0.143) | 1.51 (1.44) | 0.523 (0.338) |
| 22 | NC | 3.89 | | |
| 23 | NC | 1.22 | NC | 2.72 (1.35) |
| 24 | | 1.37 | NC | 0.384 |
| 25 | NC | 1.03 | | |
| 26 | 1.19 | 0.881 | | |
| 27 | 0.117 | 1.10 | NC | 2.17 (0.53) |
| | (n = 1) | | | |
| 39 | | | NC | 1.50 (1.18) |
| 40 | | | NC | 2.69 (0.76) |
| 41 | | | NC | 0.761 (0.497) |
| 42 | | | 0.521 | 1.47 (0.29) |
| Lansoprazole | | 0.573 (0.150) | | 0.510 (0.168) |
| 7 | 0.206 | 0.893 | NC | 1.93 (1.41) |
| 8 | NC | 1.08 | NC | 1.80 (1.20) |
| 9 | NC | 0.894 | NC | 0.341 (0.151) |
| 10 | NC | 0.989 (0.307) | | |
| 11 | NC | 0.873 (0.288) | NC | 0.933 (1.009) |
| 12 | NC | 0.931 | | |
| 13 | 0.122 | 1.77 | NC | 2.35 (1.22) |
| 14 | 0.118 | 1.39 | | 0.536 (0.217) |
| 15 | NC | 0.923 | | |
| 16 | NC | 1.00 | NC | 1.86 (0.74) |
| 17 | 1.49 | 1.13 | | |
| 18 | 0.0899 | 0.909 | | |
| 19 | 1.84 | 0.484 | | |
| 34 | | | NC | 1.11 (0.71) |
| 35 | | | NC | 1.84 (0.87) |
| 36 | | | NC | 0.389 (0.085) |
| 37 | | | NC | 2.19 (0.80) |
| 38 | | | 1.04 (0.35) | 1.43 (0.42) |
| Pantoprazole | | 0.743 | | 0.696 (0.116) |
| 28 | NC | 2.61 | NC | 1.45 (0.73) |
| 29 | NC | 0.958 | NC | 1.01 (0.30) |
| Rabeprazole | | 0.369 | | |
| 30 | 1.12 | 0.491 | | |
| 31 | 0.843 | 0.855 | | |
| 32 | 0.526 | 1.52 | | |
| 33 | 0.746 | 0.894 | | |

Values in parenthesis indicate the standard deviation, when obtained.
NC: plasma concentration of prodrug was too low to calculate half-life, or undetected.

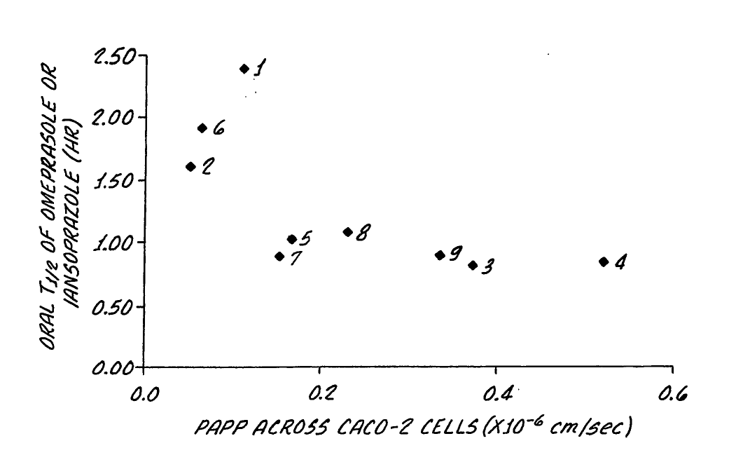

The results in Table 2D demonstrate the unexpected discovery that membrane permeability correlates with the systemic half-life of a PPI after oral administration of a PPI or a prodrug. The data also demonstrates that membrane permeability is a good predictive test for how much a given prodrug will increase the systemic half-life of a PPI because the data shows that decreasing the membrane permeability of a prodrug increases the systemic half-life of the PPI. It should be noted that there is some scatter in the data, which is believed to be due to the relatively large random error in determining the systemic half-life. However, FIG. 1 is a plot that graphically demonstrates that despite the scatter, as a general trend, systemic half-life of a PPI resulting from oral administration of its prodrug increases with decreasing membrane permeability of the prodrug. It should be noted that the correlation is not expected to be linear, since membrane permeability is a rate term associated with the reciprocal of time, whereas half-life is a measurement of time. Thus, a reciprocal relationship between the two parameters might exist, meaning that one parameter might be a function of the reciprocal value of the other. While not intending to be bound in any way by theory, these results predict that if a prodrug has lower membrane permeability than a PPI, oral administration of the prodrug will result in a longer systemic half-life of the PPI relative to the systemic half-life resulting from oral administration of the PPI itself.

TABLE 2D
Membrane permeability of proton pump inhibitors
and their prodrugs, and their systemic half-life
in dogs after their oral administration.

| | | Permeability | t_{1/2} |
|---|---|---|---|
| Compound | Parent PPI | (×10⁻⁶ cm/sec) | (hours) |
| Omeprazole | — | 13 | 0.70 |
| 1 | Omeprazole | 0.12 | 2.4 |
| 2 | Omeprazole | 0.054 | 1.6 |
| 3 | Omeprazole | 0.38 | 0.81 |
| 4 | Omeprazole | 0.52 | 0.84 |
| 5 | Omeprazole | 0.17 | 1.0 |
| 6 | Omeprazole | 0.067 | 1.9 |
| Lansoprazole | — | 15 | 0.57 |
| 7 | Lansoprazole | 0.16 | 0.89 |
| 8 | Lansoprazole | 0.23 | 1.1 |
| 9 | Lansoprazole | 0.34 | 0.89 |

### EXAMPLE 3

Capsules are prepared according to well-known commercial processes using the composition shown in Table 3.

TABLE 3

| Component | Amount (mg) |
|---|---|
| Compound 1 | 20 |
| TFF1 | 100 |
| Lactose | 200 |
| Magnesium Stearate | 3 |

### EXAMPLE 4

Capsules are prepared according to well-known commercial processes using the composition shown in Table 4.

TABLE 4

| Component | Amount (mg) |
|---|---|
| Compound 1 | 20 |
| TFF2 | 100 |
| Tamarind Seed Polysaccharide | 200 |
| Magnesium Stearate | 3 |

### EXAMPLE 5

Capsules are prepared according to well-known commercial processes using the composition shown in Table 4.

TABLE 4

| Component | Amount (mg) |
|---|---|
| Compound 1 | 20 |
| Tamarind Seed Polysaccharide | 200 |
| Magnesium Stearate | 3 |

### EXAMPLE 6

A dosage form prepared according to one of Examples 3-5 is administered orally to a person suffering from heartburn. After four days, significant relief of symptoms is observed which continues for as long as the person continues to receive the dosage form.

What is claimed is:

1. A method of preventing or treating a disease or adverse condition affecting the gastrointestinal tract comprising orally administering to a mammal
a. a therapeutically effective amount of a prodrug of a proton pump inhibitor, and
b. an effective amount of a trefoil family factor peptide, a mucoadhesive agent, or a combination thereof.

2. The method of claim 1 wherein the prodrug has a membrane permeability and the proton pump inhibitor has a membrane permeability, wherein the membrane permeability of the proton pump inhibitor is more than twice the membrane permeability of the prodrug.

3. The method of claim 2 wherein the membrane permeability of the proton pump inhibitor is more than 10 times the membrane permeability of the prodrug.

4. The method of claim 2 wherein the membrane permeability of the proton pump inhibitor is more than 100 times the membrane permeability of the prodrug.

5. The method of claim 2 wherein the membrane permeability of the proton pump inhibitor is more than 150 times the membrane permeability of the prodrug.

6. The method of claim 1 wherein the prodrug is converted to a proton pump inhibitor selected from the group consisting of omeprazole, esomeprazole, lansoprazole, pantoprazole, and rabeprazole after oral administration.

7. The method of claim 1 wherein the prodrug is converted to omeprazole after oral administration.

8. The method of claim 1 wherein the prodrug is converted to lansoprazole after oral administration.

9. The method of claim 1 wherein the prodrug comprises a sulfonyl moiety, and wherein said prodrug is converted to omeprazole after oral administration.

10. The method of claim 1 wherein the prodrug comprises a sulfonyl moiety and wherein said prodrug is converted to lansoprazole after oral administration.

11. The method of claim 1 wherein a trefoil factor family peptide is administered orally to said mammal.

12. The method of claim 1 wherein a mucoadhesive is administered orally to said mammal.

13. The method of claim 1 wherein a mucoadhesive is administered orally to said mammal, said mucoadhesive comprising Tamarind seed polysaccharide.

14. The method of claim 1 wherein a trefoil factor family peptide and a mucoadhesive are administered orally to said mammal.

15. The method of claim 1 wherein a trefoil factor family peptide and a mucoadhesive are administered orally to said mammal, and wherein said mucoadhesive comprises a polysaccharide.

16. The method of claim 1 wherein a trefoil factor family peptide and a mucoadhesive are administered orally to said mammal, and wherein said mucoadhesive comprises Tamarind seed polysaccharide.

17. A composition comprising
a prodrug of a proton pump inhibitor, and
a trefoil family factor peptide, a mucoadhesive component, or a combination thereof,
wherein said composition is suitable for use in a pharmaceutical dosage form.

18. The composition of claim 17 wherein said prodrug comprises 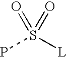
or a pharmaceutically acceptable salt thereof;
wherein
the dashed line indicates a bond that is broken systemically in said mammal;
P is a moiety that is converted systemically to a proton pump inhibitor as a result of cleavage of the bond indicated by the dashed line; and
L is a moiety which comprises a carboxylic acid.

19. The composition of claim 18 wherein L comprises a phenyl moiety.

20. The composition of claim 18 wherein P is converted systemically to a proton pump inhibitor selected from the group consisting of omeprazole, esomeprazole, lansoprazole, pantoprazole, and rabeprazole.

21. The composition of claim 18 wherein P is converted systemically to omeprazole.

22. The composition of claim 18 wherein P is converted systemically to lansoprazole.

23. The composition of claim 17 which comprises a mucoadhesive component.

24. The composition of claim 17 which comprises Tamarind seed polysaccharide.

25. The composition of claim 17 which comprises a trefoil factor family peptide.

26. The composition of claim 17 which comprises a mucoadhesive component and a trefoil factor family peptide.

27. The composition of claim 17 which comprises Tamarind seed polysaccharide and a trefoil factor family peptide.

28. The composition of claim 17 which comprises Tamarind seed polysaccharide, a trefoil factor family peptide, and further comprises a proton pump inhibitor.

29. The composition of claim 17 which further comprises a proton pump inhibitor.

30. The composition of claim 17 which comprises a mixture of prodrugs of a proton pump inhibitor.

31. The composition of claim 17 which comprises a mixture of two prodrugs of a proton pump inhibitor, said prodrugs having a membrane permeability ratio of from 2 to 1000.

32. The composition of claim 17 which comprises a mixture of two prodrugs of a proton pump inhibitor, said prodrugs having a membrane permeability ratio of from 10 to 500.

33. The composition of claim 17 which comprises a mixture of two prodrugs of a proton pump inhibitor, said prodrugs having a membrane permeability ratio of from 100 to 500.

34. An oral dosage form comprising a therapeutically active component and a trefoil factor family peptide, wherein said therapeutically active component is selected from the group consisting of proton pump inhibitors, prodrugs of proton pump inhibitors, and combinations thereof.

35. The dosage form of claim 34, wherein the therapeutically active component is omeprazole.

36. The dosage form of claim 34 wherein the therapeutically active component is esomeprazole.

37. The dosage form of claim 34 wherein the therapeutically active component is lansoprazole.

38. The dosage, form of claim 34 wherein the therapeutically active component is pantoprazole.

39. The dosage form of claim 34 wherein the therapeutically active component is rabeprazole.

40. The dosage form of claim 34 wherein the therapeutically active component comprises a prodrug of a proton pump inhibitor.

41. The dosage form of claim 34 wherein the therapeutically active component comprises both a proton pump inhibitor and a prodrug of a proton pump inhibitor.

42. The dosage form of claim 34 wherein the therapeutically active component comprises a prodrug having a sulfonyl leaving group.

43. The dosage form of claim 34 wherein the therapeutically active component comprises a prodrug having a sulfonyl leaving group, wherein said sulfonyl leaving group also comprises a carboxylic acid moiety or a pharmaceutically acceptable salt thereof.

44. The dosage form of claim 34 wherein the therapeutically active component is a single compound, said compound being a proton pump inhibitor or a prodrug of a proton pump inhibitor, which has a membrane permeability which is less than 1.4×10⁻⁵ cm/sec.

45. The dosage form of claim 34 wherein the therapeutically active component is a single compound, said compound being a proton pump inhibitor or a prodrug of a proton pump inhibitor, which has a membrane permeability which is less than 1×10⁻⁶ cm/sec.

46. The dosage form of claim 34 wherein the therapeutically active component is a single compound, said compound being a proton pump inhibitor or a prodrug of a proton pump inhibitor, which has a membrane permeability which is less than 5×10⁻⁷ cm/sec.

47. The dosage form of claim 34 wherein the therapeutically active component is a single compound, said compound being a proton pump inhibitor or a prodrug of a proton pump inhibitor which has a membrane permeability which is less than 1×10⁻⁷ cm/sec.

48. The dosage form of claim 34 wherein the therapeutically active component is a single compound, said compound being a proton pump inhibitor or a prodrug of a proton pump inhibitor which has a membrane permeability which is less than 5×10⁻⁸ cm/sec.

49. The dosage form of claim 34 wherein the trefoil factor family peptide is TFF1, TFF2, or TFF3.

50. The dosage form of claim 34 wherein the trefoil factor family peptide is TFF1 or TFF2.

51. The dosage form of claim 34 wherein the trefoil factor family peptide is TFF1.

52. The dosage form of claim 34 wherein the trefoil factor family peptide is TFF2.

53. The dosage form of claim 34 which further comprises a mucoadhesive.

54. The dosage form of claim 34 which further comprises Tamarind seed polysaccharide.

55. A method of preventing or treating a disease or adverse condition comprising administering directly into a gastrointestinal tract of a mammal an effective amount of a therapeutically active agent, and
a therapeutically effective amount of a trefoil factor family peptide, wherein
said therapeutically active agent comprises a compound which, when administered orally, results in inhibition of the gastric H,K-ATPase enzyme, and
wherein said disease or condition affects the gastrointestinal tract.

56. The method of claim 55 wherein the therapeutically active agent comprises a benzimidazole derivative.

57. The method of claim 55 wherein the therapeutically active agent comprises a benzimidazole derivative and a biological leaving group.

58. The method of claim 55 wherein the therapeutically active agent comprises a benzimidazole derivative and a biological leaving group with a sulfonyl moiety.

59. The method of claim 55 wherein the therapeutically active agent comprises a benzimidazole derivative and a biological leaving group with a sulfonyl moiety, said biological leaving group further comprising a carboxylic acid or a pharmaceutically acceptable salt thereof.

60. The method of claim 55 wherein the therapeutically active agent is a proton pump inhibitor or a salt or prodrug thereof, wherein said proton pump inhibitor is selected from the group consisting of omeprazole, esomeprazole, lansoprazole, pantoprazole, and rabeprazole.

61. The method of claim 55 wherein the therapeutically active agent is a prodrug of a proton pump inhibitor wherein said proton pump inhibitor is selected from the group consisting of omeprazole, esomeprazole, lansoprazole, pantoprazole, and rabeprazole.

62. The method of claim 55 wherein the therapeutically active agent comprises a mixture of a proton pump inhibitor and its prodrug.

63. The method of claim 55 wherein the trefoil family factor peptide is TFF1.

64. The method of claim 55 wherein the trefoil family factor peptide is TFF2.

65. The method of claim 55 wherein the trefoil family factor peptide is TFF3.

66. The method of claim 55 wherein a mucoadhesive is also administered to said mammal.

67. The method of claim 55 wherein Tamarind seed polysaccharide is also administered to said mammal.

68. The method of claim 55 wherein the therapeutically active agent comprises a mixture of a proton pump inhibitor and a prodrug of said proton pump inhibitor, said proton pump inhibitor having a membrane permeability and said prodrug having a membrane permeability, wherein the membrane permeability of the proton pump inhibitor is more than 10 times the membrane permeability of the prodrug.

69. The method of claim 68 wherein the membrane permeability of the proton pump inhibitor is more than 100 times that of the prodrug.

70. The method of claim 68 wherein the membrane permeability of the proton pump inhibitor is more than 150 times that of the prodrug.
